(19) United States
(12) Patent Application Publication
(10) Pub. No.: US 2008/0152077 A1
(43) Pub. Date: Jun. 26, 2008

(54) MAMMOGRAPHY TOMOSYNTHESIS APPARATUS AND METHOD

(76) Inventor: Martin Ramsauer, Pyrbaum (DE)

Correspondence Address: SCHIFF HARDIN, LLP;PATENT DEPARTMENT, 6600 SEARS TOWER, CHICAGO, IL, 60606-6473, US

(21) Appl. No.: 11/958,561

(22) Filed: Dec. 18, 2007

(30) Foreign Application Priority Data
Dec. 18, 2006 (DE) 10 2006 059 740.0

(51) Int. Cl. A61B 6/04

(52) U.S. Cl. 378/37

(57) ABSTRACT

Mammography apparatus, in particular for tomosynthesis, has a subject table extending in an X-Y plane, an x-ray radiator, a detector arranged opposite the x-ray radiator, held (supported by) and a compression element a holder. The x-ray radiator is supported such that it can pivot in an X-Z plane around a central axis proceeding parallel to the Y-direction. In order to be able to acquire interference-free exposures of a subject to be examined, the compression element exhibits a length in the X-direction that is greater than the extent of the detector in the X-direction, such that the holder of the compression element casts no shadows in the x-ray exposure.

### BACKGROUND OF THE INVENTION

1. Field of the Invention

The invention concerns a mammography apparatus, in particular for tomosynthesis.

2. Description of the Prior Art

Medical examinations of the soft tissue of the human breast with x-ray radiation are implemented with a mammography apparatus, such examinations in particular serve for early detection of breast cancer. The breast to be examined is clamped between a subject table and a compression element that can be displaced against the subject table, the compression element being fashioned as a type of plate. The compression element is fashioned from a material permeable to x-ray radiation and is held by a holder (mounting; bracket) that, with the compression element, forms a compression unit and is impermeable for x-ray radiation. The holder is supported such that it can be displaced vertically on a stand of the mammography apparatus. An x-ray examination is subsequently implemented with an irradiation unit fashioned as an x-ray radiator. An x-ray detector that is struck by an x-ray beam emitted from the irradiation unit is typically integrated into the subject table. Soft x-ray radiation in the range below 50 kV, in particular below 30 kV, is used.

Tomosynthesis is an examination method making use of a digital mammography apparatus in which “virtual” 3D information is acquired that is provided in the form of reconstructed 2D slices. In a tomosynthesis examination the irradiation unit moves continuously through a relatively large angle range, for example an angle range of +/−25° around a horizontal central axis given a stationary subject table.

### SUMMARY OF THE INVENTION

An object of the present invention is to provide a mammography apparatus (in particular for tomosynthesis) with which interference-free exposures of a subject to be examined are generated.

The object is inventively achieved by a mammography apparatus (in particular for tomosynthesis) having a subject table extending in an X-Y plane, an x-ray radiator, a detector arranged opposite the x-ray radiator and a compression element that is permeable to x-ray radiation, wherein the x-ray radiator is supported so as to pivot in the X-Z plane around a central axis proceeding parallel to the Y-direction; and wherein the compression element exhibits a length in the X-direction that is greater than the extent of the detector in the X-direction.

The invention is based on the insight that interference-free exposures with a high quality can be acquired by a mammography apparatus, even with a relatively large deflection of the x-ray radiator around the central axis, by fashioning the compression element to be so long that, in the operation of the mammography apparatus, the x-ray beam only passes through the compression element (which is permeable for x-rays). Due to the length of the compression element, the x-ray radiation is prevented from registering the holder that is fashioned from a material that impermeable for x-ray radiation, and thus would negatively affect the quality of the x-ray exposures. Insofar as x-ray radiation strikes the holder, this appears as a shadow in the x-ray exposure. Given a stationary detector, the length of the compression element can be a function of the predetermined length of the detector, the deflection angle of the x-ray radiator and the height of the compression element above the detector, so the determination of the necessary length of the compression element can ensue without a significant computational effort.

In a preferred embodiment, starting from a zero position, the x-ray radiator can be pivoted toward both sides up to respective, predetermined deflection positions, with the length of the compression element being selected such that in the deflection positions of the x-ray radiator the entire x-ray beam penetrates the compression element. A particularly high quality of the x-ray exposure is achieved in this embodiment, since for each irradiation angle along the movement path of the x-ray beam it is ensured that the holder of the compression element remains outside of the range of the x-ray beam.

The x-ray radiator in the deflection positions appropriately encompasses a deflection angle of +/−25° with the Z-direction. A deflection angle of +/−25° is typically used in a tomosynthesis examination.

In cross-section in the X-Z plane, the compression element is preferably fashioned in the form of a trapezoid tapering toward the detector. Such a compression element exhibits the shape of a trough having an underside forming an essentially rectangular compression plate around which is arranged an edge that is bent outwardly. The angled position of the edge prevents, given irradiation from the deflection positions, a portion of the x-ray beam from first penetrating the edge and then the compression plate so that the edge of the compression element would cast a shadow on the image exposure acquired by the detector. In this embodiment of the compression element the surface of the compression plate can be advantageously kept small; it in particular corresponds to the length of the compression plate in the X-direction of the detector. This enables particularly good accessibility for positioning the breast to be examined.

Edge sides of the trapezoidal compression element advantageously enclose an angle with the Z-direction in the range between 10° and 15°, in particular 12°. An angled position of the edge sides relative to the vertical Z-direction in this angle range has proven to be sufficient in order to prevent a shadow of the edge being cast in an image acquisition from one of the deflection positions.

In a preferred embodiment, a coordinate system is shown on the compression element. This is visible in the generated x-ray image and serves for determination of the coordinates of a tumor. For example, this is advantageous in a tomosynthesis examination. In such an examination, “virtual” 3D information of the breast is obtained by a reconstruction of the acquired image data. A number of slices can be presented situated atop one another so that information about the Z-position, i.e. the depth of the tumor, is present. Information about the position of the tumor in the X-Y plane is additionally acquired by means of the perpendicular acquisition in the zero position and with the aid of the superimposed coordinate system.

According to a further preferred embodiment, the compression plate of the compression element, the compression plate being arranged in the X-Y plane, is provided with a whole pattern. In this embodiment, after the determination of the 3D position of the tumor the examination can be extended by an extraction of tissue material (biopsy), in which a physician can extract tissue through the openings of the compression plate by means of a biopsy needle. This is particularly advantageous for biopsies in the framework of tomosynthesis examination.

In order to achieve an exceptional image quality of the exposures acquired with the mammography apparatus, the detector is advantageously fashioned for digital acquisitions. A flat detector thus is provided for use in the mammography apparatus, this detector having a photoconductor that absorbs the x-ray radiation and generates electrical signals without intermediate components.

The compression element appropriately alternately exhibits a height of 40 mm or 80-90 mm. The height of the compression element is defined by the height of the edge that extends over the compression plate.

The field of application of the mammography apparatus can furthermore be expanded by supporting the subject table so that it can pivot around the central axis. An examination known as an MLO examination is thereby possible. In such an examination, the subject table follows the x-ray radiator so that the subject table and the x-ray radiator are always aligned in the same position and at the same distance relative to one another.

The above object is furthermore inventively achieved by a method for imaging using the above-described mammography apparatus, wherein the x-ray radiator is moved between two deflection positions along a travel path and a number of exposures are made, and wherein the length of the compression element is selected such that the entire x-ray beam penetrates the compression element in all angle positions along the travel path of the x-ray radiator. An extensive image data set is thereby acquired with which a precise diagnosis is possible. The advantages and preferred embodiments mentioned with regard to the mammography apparatus are applicable to the method as well.

### BRIEF DESCRIPTION OF THE DRAWINGS

FIG. 1 is a side view of a mammography apparatus.

FIG. 2 is a front view of the mammography apparatus of FIG. 1.

FIG. 3 illustrates two deflection positions during irradiation by means of a mammography apparatus in a tomosynthesis examination.

FIG. 4a,b is a perspective view and a plan view of a compression element.

FIG. 5 is a front view of the compression element according to FIG. 4.

### DESCRIPTION OF THE PREFERRED EMBODIMENTS

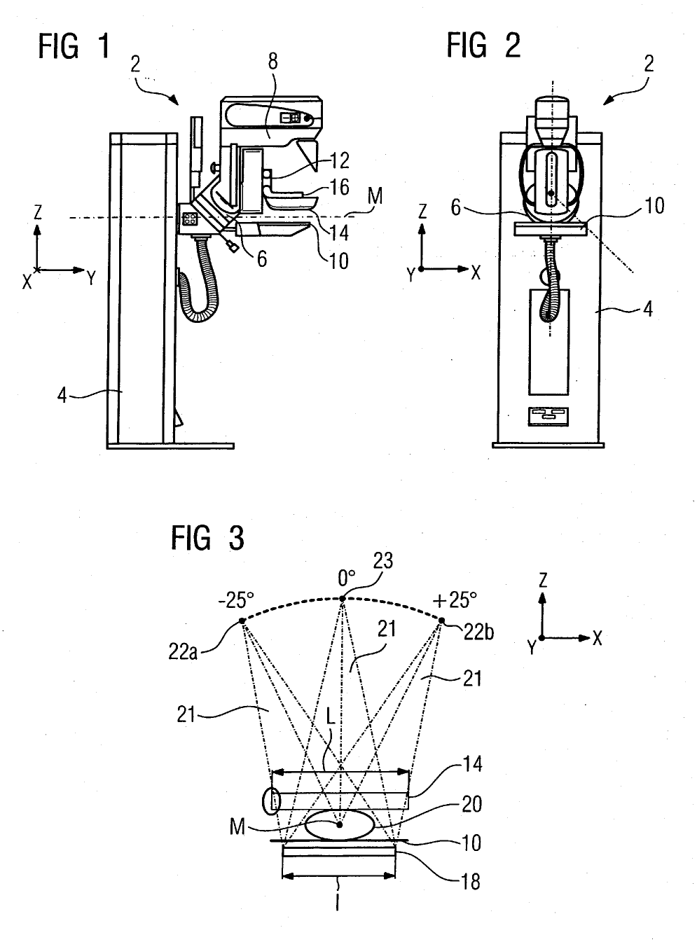

In FIGS. 1 and 2 a side view and a front view of a mammography apparatus 2 are shown. The mammography apparatus 2 has a base body fashioned as a stand 4 and a curving apparatus arm 6 projecting from this stand 4, at the free end of which apparatus arm 6 is arranged an irradiation unit 8 fashioned as an x-ray radiator. Furthermore, a subject table 10 and a compression unit 12 are borne on the apparatus arm 6. The compression unit 12 has a compression element 14 that is arranged such that it can be displaced along a vertical Z-direction relative to the subject table 10, as well as a holder 16 for the compression element 14. A type of lift guidance in the compression unit 12 is hereby provided for movement of the holder 16 together with the compression element 14. Furthermore, a detector 18 (see FIG. 3) that, in this exemplary embodiment, is a digital detector, is arranged in a lower region of the subject table 10.

The mammography apparatus 2 is in particular provided for tomosynthesis examinations in which the radiation unit 8 is moved through a relatively large angle range around a central axis M proceeding parallel to the Y-direction, as is clear from FIG. 3. Multiple slice exposures of the subject 20 to be examined (which subject 20 is held stationary between the subject table 10 and the compression element 14) are thereby acquired. Given the image acquisitions from the different angle positions, an x-ray beam 21 (which x-ray beam 21 is fan-shaped in cross-section) penetrates the compression element 14, the subject 20 to be examined and the subject table 10 and strikes the detector 18. The detector 18 is dimensioned such that the image acquisitions can be made in an angle range between two deflection positions 22a, 22b given corresponding deflection angles of +/−25°. The deflection positions 22a, 22b are arranged on both sides of a zero position 23 in the X-Z plane, in which zero position 23 the x-ray beam 21 vertically strikes the detector 18. In this exemplary embodiment the flat detector 18 in particular exhibits a size of 24×30 cm.

Due to the relatively large pivot range of the irradiation unit 8, the length (L) of the compression element 14 in the X-direction is selected greater than the length (I) of the detector 18 in the same direction. It is thus prevented that, given irradiation of the subject 20 to be examined from the deflection positions 22a, 22b, the holder 16 (which is impermeable to the x-ray beam 21) of the compression element 14 is acquired and imaged by the x-ray beam 21, which would have a negative effect on the quality of the image exposure.

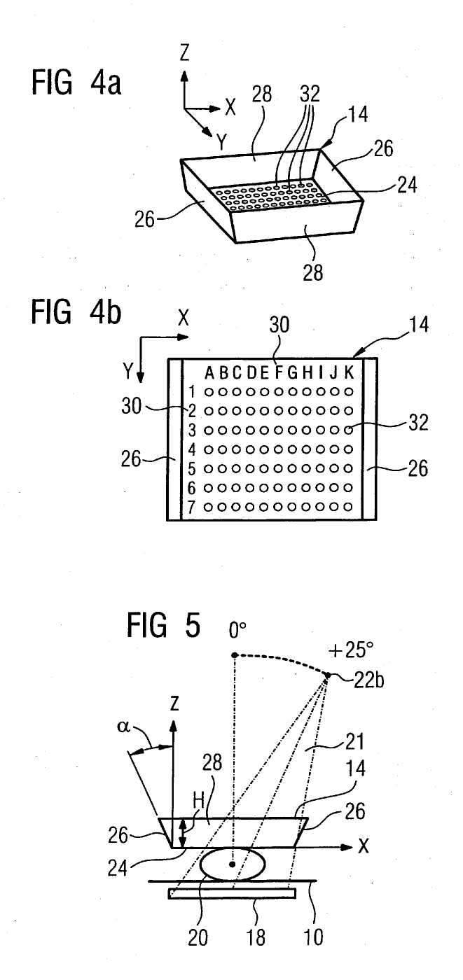

In the exemplary embodiment according to FIG. 4a the compression element 14 in cross-section in the X-Z plane is fashioned as a type of trapezoid tapering towards the detector 18. The compression element 14 is trough-shaped and comprises a rectangular compression plate 24 around whose circumference are arranged four bent edge sides 26, 28. In the present exemplary embodiment the two opposite edge sides 28 that border the sides of the compression plate 24 extending in the X-direction stand perpendicular to the plane of the compression plate 24. The other two edge sides 26 are fashioned at an angle relative to the plane of the compression plate 24. It is hereby prevented that, given an irradiation from the deflection positions 22a, 22b, the edge sides 26 are acquired by the x-ray beam 21 and thus cast a shadow on the exposure. As is apparent from the plan view according to FIG. 4b, the compression element 14 is provided with a coordinate system 30, i.e. a scaling that is impermeable for digital x-ray receiver radiation and that is visible on the x-ray exposures is applied on the compression element 14. Such a compression element 14 is in particular used given a biopsy examination in order to determine the position of a finding in the subject 20 with high precision. The compression plate 24 is hereby additionally provided with holes 32 so that a tissue extraction can be implemented without delay after the localization of the finding.

As is to be seen from FIG. 5, the two edge sides 26 with the Z-direction enclose an angle α that in particular lies in the range between 10° and 15° and in this exemplary embodiment is 12°. The height of the edge sides 26, 28 can be selected differently. Moreover, the height H of the compression element can be selected differently; the edge sides 26, 28 in particular alternately exhibit a height of 40 mm or 80-90 mm above the compression plate 24.

Moreover, to expand the function of the mammography apparatus 2, the entire apparatus arm 6 can be supported (mounted) such that it can be pivoted in the X-Z plane without it being moved. In this embodiment an MLO examination (acquisition from an angle of +/−45°) is possible in which the irradiation unit 8 is always aligned in the same orientation relative to the subject table 10. The compression unit 12 that is permanently connected with the apparatus arm 6 is likewise pivoted as well, such that its position relative to the subject table 10 does not change.

Although modifications and changes may be suggested by those skilled in the art, it is the intention of the inventor to embody within the patent warranted hereon all changes and modifications as reasonably and properly come within the scope of his contribution to the art.

I claim as my invention:

1. A mammography tomosynthesis apparatus comprising:
a subject table configured to support a subject thereon, said subject table extending in an X-Y plane of a coordinate system having an X-direction, a Y-direction and a Z-direction that are orthogonal relative to each other;
a compression element configured to compress the subject on the subject table;
an x-ray radiator that emits an x-ray beam that irradiates the subject on the subject table while compressed by the compression element;
a support that supports the x-ray radiator to allow the x-ray radiator to pivot in an X-Z plane around a central axis that proceeds parallel to the Y-direction;
a detector disposed opposite the x-ray radiator that detects x-rays in said x-ray beam, said detector having a detector extent in the X-direction; and
said compression element having a length in the X-direction that is greater than said detector extent in the X-direction.

2. A mammography tomosynthesis apparatus as claimed in claim 1 wherein said support, starting from a zero position, allows the x-ray radiator to be pivoted on opposite sides of said zero position to respective predetermined deflection positions, and wherein said length of said compression element is selected so that, at each of said respective deflection positions, said x-ray beam complete penetrates the compression element.

3. A mammography tomosynthesis apparatus as claimed in claim 2 wherein said respective deflection positions are at +/−25° with respect to said zero position, in the Z-direction.

4. A mammography tomosynthesis apparatus as claimed in claim 1 wherein said compression element has a cross-section in the X-Z plane forming a trapezoid that tapers toward the detector.

5. A mammography tomosynthesis apparatus as claimed in claim 4 wherein said compression element has edges of said trapezoidal cross-section enclosing an angle in the Z-direction in a range between 10° and 15°.

6. A mammography tomosynthesis apparatus as claimed in claim 5 wherein said edge sides form an angle of 12°.

7. A mammography tomosynthesis apparatus as claimed in claim 1 wherein said compression element has a planar coordinate system applied thereon comprising coordinate system markings that are impermeable to said x-ray beam and that are visible in an image generated by said detector.

8. A mammography tomosynthesis system as claimed in claim 6 wherein said compression plate extends in the X-Y plane, and comprises a hole pattern therein conforming to said coordinate system.

9. A mammography tomosynthesis apparatus as claimed in claim 1 wherein said detector is a digital detector.

10. A mammography tomosynthesis apparatus as claimed in claim 1 wherein said compression element has a thickness in a direction of propagation of said x-ray beam therethrough of 40 mm.

11. A mammography tomosynthesis apparatus as claimed in claim 1 wherein said compression element has a thickness in a direction of propagation of said x-ray beam therethrough in a range between 80 and 90 mm.

12. A mammography tomosynthesis apparatus as claimed in claim 1 comprising a support that supports the subject table allowing the subject table to pivot around said central axis.

13. A method for imaging a female breast, comprising the steps of:
supporting a female breast on a support table;
irradiating the breast on the support table by moving an x-ray source between two deflection positions along a travel path to obtain a plurality of exposures of the breast from different positions along said travel path; and
while irradiating the breast with said x-ray beam, compressing the breast on the support plate with a compression element having a length that causes an entirety of the x-ray beam to penetrate the compression element at all positions along said travel path.
